SEQUENCE LISTING (9 sequences)

SEQ ID NO: 1 - protein, 7 aa, synthetic construct
VHPKQHR

SEQ ID NO: 2 - protein, 7 aa, synthetic construct
QAHPHVD

SEQ ID NO: 3 - protein, 12 aa, synthetic construct
DITWDQLWDLMK

SEQ ID NO: 4 - protein, 8 aa, synthetic construct
CSRNLIDC

SEQ ID NO: 5 - protein, 8 aa, synthetic construct
CTSDHAVC

SEQ ID NO: 6 - protein, 7 aa, synthetic construct
  MOD_RES 1: note = DSPE-PEG on the N-terminal of the first residue
VHPKQHR

SEQ ID NO: 7 - protein, 12 aa, synthetic construct
  MOD_RES 1: note = DSPE-PEG on the N-terminal of the first residue
DITWDQLWDLMK

SEQ ID NO: 8 - protein, 9 aa, synthetic construct
CTSGTHPRC

SEQ ID NO: 9 - protein, 9 aa, synthetic construct
  MOD_RES 1: note = DSPE-PEG on the N-terminal of the first residue
CTSGTHPRC

(19) United States
(12) Patent Application Publication
(10) Pub. No.: US 2026/0263627 A1
(43) Pub. Date: Sep. 10, 2026

(54) Targeted Nanomedicine for Treating Lung Disorders

(71) Applicant: The University of Chicago, Chicago, IL (US)

(72) Inventors: Yun Fang, Chicago, IL (US); Matthew Tirrell, Chicago, IL (US); Zhengjie Zhou, Chicago, IL (US)

(21) Appl. No.: 18/875,541

(22) PCT Filed: Jun. 16, 2023
(86) PCT No.: PCT/US2023/068600

§ 371 Date: Dec. 16, 2024

Related U.S. Application Data
(60) Provisional application No. 63/353,467, filed on Jun. 17, 2022.

(51) Int. Cl. A61K 47/69; A61K 9/00; A61K 38/17; A61K 38/45; A61K 47/62; A61P 11/00

(52) U.S. Cl. CPC A61K 47/6911; A61K 9/0019; A61K 38/1709; A61K 38/45; A61K 47/62; A61K 47/6929; A61P 11/00; C12Y 207/07

(57) ABSTRACT

This disclosure relates to compositions and methods for treating lung disorders, including, for example, Acute Respiratory Distress Syndrome (ARDS), Ventila-tor-Induced Lung Injury (VILI), Acute lung injury (ALI), and other acute and chronic lung disorders. Further, wherein a lipid nanoparticle, comprises: a) a VCAM-1 targeting molecule; and b) an RNA molecule encoding Kriippel-like Factor 2 (KLF2).

### CROSS REFERENCE TO RELATED APPLICATIONS

This application claims the benefit of U.S. Provisional Application No. 63/353,467, filed Jun. 17, 2022, which is incorporated by reference herein in its entirety.

### STATEMENT REGARDING FEDERALLY SPONSORED RESEARCH

This invention was made with government support under grant numbers R01 HL159558-01A1 and R35 HL161244-01 funded by the National Institutes of Health (NIH). The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jun. 16, 2023, is named “22-0856-WO_SequenceListing.xml” and is 10,002 bytes in size.

### BACKGROUND OF THE DISCLOSURE

### Field of Invention

This disclosure relates to compositions and methods for treating lung disorders, including, for example, Acute Respiratory Distress Syndrome (ARDS), Ventilator-Induced Lung Injury (VILI), Acute lung injury (ALI), and other acute and chronic lung disorders.

### Technical Background

Acute Respiratory Distress Syndrome (ARDS) is a severe form of lung injury, in which the alveolar-vascular barrier breaks down, causing vascular exudate to flood the breathing units of the lung, resulting in hypoxia and the need for advanced respiratory support which has been demonstrated by Matthay et al. (J. Clin. Investig., 2012, 122 (8), 2731-2740). ARDS is the major cause of death in critically ill patients suffering from influenza or SARS-COV-2 viral infections. ARDS affects 200,000 people resulting in 3.6 million hospital days and 75,000 deaths annually in the U.S. despite improvement in supportive care listed by Rubenfield et al. (N. Engl. J. Med., 2005, 353 (16), 1685-1693). These numbers do not reflect the latest COVID-19 pandemic, which has greatly increased the number. Influenza associated ARDS during the 2009 pandemic was the leading cause of hospitalization and intensive case utilization, and led to mortality of 10-20% among the critically ill patients. Currently, pandemic COVID-19 has an estimated mortality between 35 and 50% for those on mechanical ventilation in the U.S. There are already more than 1,000,000 deaths in the U.S. by May 2022 due to COVID-19. Respiratory support by itself in the form of mechanical ventilation can worsen ARDS through ventilator-induced lung injury (VILI) as shown in Slutsky et al. (N. Engl. J. Med. 2013, 369 (22), 2126-2136).

While new COVID-19 treatments have improved mortality, there are still excess ARDS deaths. Acute lung injury (ALI) is the animal experimental correlate of ARDS. Endothelial barrier disruption and uncontrolled cytokine storm are major molecular signatures causing influenza and COVID-19 ARDS. Endothelial dysfunction also contributes significantly to the uncontrolled cytokine storm in ARDS as shown in Teijaro et al. (Cell 2011, 146 (6), 980-991). Current ARDS treatments do not directly target diseased blood vessels, underscoring an unmet medical need heightened by the COVID-19 pandemic. Pulmonary endothelial dysfunction, characterized by elevated inflammation and increased monolayer permeability, results in parenchymal accumulation of leukocytes, protein, and extravascular water, leading to pulmonary edema, ischemia, and coagulation associated with COVID-19. In agreement with these findings, endothelial dysfunction was reported in autopsies of influenza and COVID-19 patients. Promoting endothelial health therefore is an attractive approach to reduce ARDS.

Pulmonary epithelial cells, the primary target of respiratory viruses such as influenza or SARS-COV-2, play a pivotal role in initiating innate immune responses to defend against virus infection. Viral infections lead to epithelial cell dysfunction, causing increased paracellular permeability, a hallmark of ARDS. 2′-5′-oligoadenylatesynthetase 1 (OAS1) exerts antiviral response through a classical OAS/RNase L pathway to degrade viral RNA. Moreover, OAS1 was shown to have a robust anti-SARS-COV-2 activity by binding the conserved stem loop of virus. Enhancing OAS1 in epithelium can lessen virus-induced lung injury.

Currently, there are few pharmacological treatments that directly target ARDS and VILI, underscoring unmet medical needs in a heightened state due to the COVID-19 pandemic. ARDS and VILI are underserved by the nanomedicine community. There is a need for a convenient therapeutic avenue to treat ARDS as the worldwide COVID-19 pandemic leads to potentially hundreds of thousands of individuals with fibroproliferative ARDS. Therefore, targeted nanomedicine approaches with tremendous potential to treat ARDS were developed as described herein.

### SUMMARY OF THE DISCLOSURE

This disclosure describes compositions and methods for treating lung disorders, including, for example, Acute Respiratory Distress Syndrome (ARDS), Ventilator-Induced Lung Injury (VILI), Acute lung injury (ALI), and other acute and chronic lung disorders.

In a first aspect, the present disclosure provides a lipid nanoparticle, comprising a VCAM-1 targeting molecule; and an RNA molecule encoding Krüppel-like Factor 2 (KLF2).

In some embodiments of the first aspect, the lipid nanoparticle comprises a polyamidoamine (PAMAM) dendrimer (G0-C14), cholesterol, polyethylene glycol 2000 (PEG), 1,2-Distearoyl-sn-glycero-3-phosphoethanolamine-Poly(ethylene glycol) (DSPE-PEG), dioleoylphospha-tidylethanolamine (DOPE), and Krüppel-like Factor 2 (KLF2) mRNA encapsulated by the lipid nanoparticle. In some embodiments, the PEG domain comprises PEG having an average molecular weight of about 1,000 to about 100,000 Daltons. In some embodiments, the VCAM-1 targeting molecule comprises a peptide comprising the amino acid sequence VHPKQHR (SEQ ID NO: 1) or DITWDQLWDLMK (SEQ ID NO: 3). In some embodiments, the VCAM-1 targeted lipid nanoparticle comprises DSPE-PEG-VHPKQHR (SEQ ID NO:6) or DSPE-PEG-DITWDQLWDLMK (SEQ ID NO:7). In some embodiments, the RNA molecule encoding KLF2 mRNA. In some embodiments, the KLF2 mRNA is a ml Ψ-substituted KLF2 mRNA. In some embodiments, the RNA molecule encoding KLF2 mRNA is at a concentration of about 0.01 μM to about 100 μM. In some embodiments, the RNA molecule encoding KLF2 mRNA is at a concentration of about 2 μM. In some embodiments, the lipid nanoparticle comprises a molar ratio of the polyamidoamine (PAMAM) dendrimer (G0-C14) to DSPE-PEG is about 2:1 to about 15:1; cholesterol to DSPE-PEG is about 15:1 to about 40:1; and DOPE to DSPE-PEG is about 15:1 to about 30:1. In some embodiments, the lipid nanoparticle comprises molar ratios of PAMAMG0-C14:DOPE:Cholesterol:DSPE-PEG of 5:15:25:1.

In a second aspect, the present disclosure provides a pharmaceutical composition, comprising a therapeutically effective amount of a lipid nanoparticle comprising a VCAM-1 targeting molecule and an RNA molecule encoding KLF2, and a pharmaceutically acceptable carrier, solvent, adjuvant, and/or diluent. In some embodiments, the pharmaceutical composition is formulated for oral, intravenous, topical, ocular, buccal, systemic, nasal, injection, transdermal, rectal, or vaginal administration. In some embodiments, the pharmaceutical composition is formulated for inhalation or insufflation.

In a third aspect, the present disclosure provides a pharmaceutical composition for pulmonary delivery of RNA, comprising a lipid nanoparticle comprising a VCAM-1 targeting molecule, and an RNA molecule encoding KLF2, and pharmaceutically acceptable carrier, wherein the composition is formulated such that once administered to the lung, it results in the delivery of the RNA molecule to a lung cells.

In a fourth aspect, the disclosure provides a method of treating a lung disorder in a subject, comprising administering to the subject a therapeutically effective amount of a pharmaceutical composition comprising a VCAM-1 targeting molecule and an RNA molecule encoding KLF2, wherein the targeted lipid nanoparticle is preferentially targeted to inflamed endothelial cells associated with the lung disorder and reducing inflammation at the site of the inflamed endothelial cells. In some embodiments of the fourth aspect, the lung disorder comprises one or more of Acute Respiratory Distress Syndrome (ARDS), Acute Lung Injury (ALI), or ventilator-induced lung injury (VILI). In some embodiments of the fourth aspect, the method results in one or more of an enhancement of KLF2 mRNA and reduction in viral titers at the site of the inflamed endothelial cells compared to a control. In some embodiments, the method results in one or more of an increase of at least about 2 fold to at least about 1000 fold of the RNA encoding KLF2 compared to a control, an increase of at least about 2 fold to at least about 200 fold of KLF2 protein compared to a control, or a reduction in viral titers compared to a control at the site of the inflamed endothelial cells. In certain embodiments, the method further includes stimulating endothelial growth at the site of the wound. In some embodiments, the probability of survival of the individual is at least about 10% greater than an expected probability of survival without administration of the pharmaceutical composition.

In a fifth aspect, the disclosure provides a lipid nanoparticle, comprising an epithelium targeting molecule; and an RNA molecule encoding 2′-5′-oligoadenylatesynthetase 1 (OAS1). In some embodiments, the lipid nanoparticle comprises a polyamidoamine (PAMAM) dendrimer (G0-C14), cholesterol, polyethylene glycol 2000 (PEG),1,2-Distearoyl-sn-glycero-3-phosphoethanolamine-Poly(ethylene glycol) (DSPE-PEG), and dioleoylphospha-tidylethanolamine (DOPE), and wherein the lipid nanoparticle encapsulates the RNA molecule encoding OAS1. In some embodimnets, the DSPE-PEG comprises a PEG domain comprising PEG having an average molecular weight of about 1,000 to about 100,000 Daltons. In some embodiments of the fifth aspect, the epithelium targeting molecule comprises a peptide comprising the amino acid sequence CTSGTHPRC (SEQ ID NO: 8). In some embodiments, the epithelium targeted lipid nanoparticle comprises DSPE-PEG-CTSGTHPRC (SEQ ID NO: 9). In some embodiments of the fifth aspect, the RNA molecule encoding OAS1 is mRNA. In some embodiments of the fifth aspect, the OAS1 mRNA is a m1Ψ-substituted OAS1 mRNA. In some embodiments, the lipid nanoparticle encapsulates about 0.01 μM to about 100 μM of the RNA molecule encoding OAS1. In some embodiments of the fifth aspect, the molar ratio of the polyamidoamine (PAMAM) dendrimer (G0-C14) to DSPE-PEG is about 2:1 to about 15:1; cholesterol to DSPE-PEG is about 15:1 to about 40:1; and DOPE to DSPE-PEG is about 15:1 to about 30:1.

In a sixth aspect, this disclosure provides a pharmaceutical composition, comprising a therapeutically effective amount of a lipid nanoparticle comprising a epithelium targeting molecule and an RNA molecule encoding OAS1; and a pharmaceutically acceptable carrier, solvent, adjuvant, and/or diluent. In some embodiments, the pharmaceutical composition is formulated for inhalation, insufflation, oral, intravenous, topical, ocular, buccal, systemic, nasal, injection, transdermal, rectal, or vaginal administration. In some embodiments, the pharmaceutical composition is formulated for inhalation or insufflation.

In a seventh aspect, this disclosure provides, a pharmaceutical composition for pulmonary delivery of RNA comprising a lipid nanoparticle comprising a epithelium targeting molecule; and an RNA molecule encoding OAS1; and a pharmaceutically acceptable carrier, wherein the composition is formulated such that once administered to the lung, it results in the delivery of the RNA molecule to a lung cell.

In an eighth aspect, this disclosure provides, a method of treating a lung disorder in a subject, comprising: a) administering to the subject a therapeutically effective amount of a pharmaceutical composition comprising a lipid nanoparticle comprising a epithelium targeting molecule and an RNA molecule encoding OAS1, wherein the lipid nanoparticle is preferentially targeted to inflamed endothelial cells associated with the lung disorder; and b) reducing inflammation at the site of the inflamed endothelial cells. In some embodiments, the lung disorder comprises one or more of Acute Respiratory Distress Syndrome (ARDS), Acute Lung Injury (ALI), or Ventilator-Induced Lung Injury (VILI). In some embodiments, the method results in one or more of an increase of at least about 2 fold to at least about 1000 fold of the RNA encoding OASIcompared to a control, an increase of at least about 2 fold to at least about 2000 fold of OAS1protein compared to a control, or a reduction in viral titers compared to a control at the site of the inflamed endothelial cells. In some embodiments, the probability of survival of the subject is at least about 10% greater than an expected probability of survival without administration of the pharmaceutical composition.

In certain embodiments, the lipid nanoparticle compositions can be formulated to target additional lung cells. For instance, the lipid nanoparticle can be engineered to display the peptide CTSGTHPRC (SEQ ID NO: 8) which binds epithelium. Epithelium-targeting lipid nanoparticle can be used to deliver mRNA of ani-viral gene OAS1, a sequence described by Morris et al. (J. Control. 2011, 151 (1), 83-94).

In certain embodiments, the lipid nanoparticle compositions disclosed herein can be administered to a subject having respiratory distress syndrome/acute respiratory distress syndrome (ARDS) induced by influenza or SARS-COV-2 viruses. In some embodiments, the lipid nanoparticle compositions disclosed herein reduce respiratory distress syndrome/acute respiratory distress syndrome (ARDS) induced by influenza or SARS-COV-2 viruses in the patient.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-1C show reduced KLF2 levels in distressed lungs. KLF2 reduction in mouse lungs subjected to H1N1 or SARS-COV-2 viruses (FIG. 1A; n=5-6, representative images shown). Data are mean±SEM. Expression KLF2 is significantly reduced in the mouse lung subjected to acute respiratory distress syndrome (ARDS) induced by intratracheal delivery of lipopolysaccharides (LPS) (FIG. 1B). Expression of KLF2 is significantly reduced in the rat lung subjected to acute respiratory distress syndrome (ARDS) and ventilator-induced lung injury (VILI) stimulated by intratracheal delivery of LPS along with high-tidal volume ventilation (HTV), when compared to those subjected to LPS and low-tidal volume ventilation (LTV) (FIG. 1C).

FIG. 2 shows that KLF2 protein levels are significantly reduced in endothelium cells in lungs from COVID-19 patients when compared to healthy human lungs. This data are described in Wu et al. (Am. J. Respir., 2021, 65 (2), 222-226) (n=5-10). Data are mean±SEM.

FIG. 3. LPS-induced cell leak in mouse alveolar space was markedly reduced by prophylactic KLF2 overexpression (OE) using non-targeting polyethylenimine nanoparticles (PEI NP) (n=5). These data are described in Huang et al. (Am. J. Respir. Crit., 2017, 195 (5), 639-651). Data are mean±SEM.

FIG. 4 shows a novel VCAM1-targeting lipid nanoparticle that was engineered to effectively encapsulate and deliver messenger RNA (mRNA) of KLF2 to activated endothelial cells. VCAM1-targeting lipid nanoparticles were characterized by transmission electron microscopy (TEM) and dynamic light scattering (DLS).

FIG. 5 shows that the VCAM-1 targeting lipid nanoparticle effectively delivered functional mScarlet mRNA to inflamed pulmonary microvascular endothelial cells in vitro and in vivo. (n=5, representative data/images shown).

FIGS. 6A-6B show KLF2 mRNA encapsulated, VCAM1-targeting lipid nanoparticles effectively deliver KLF2 mRNA to inflamed lung in vivo (mice) and reduce lung inflammation induced by SARS-COV-2 viruses. (FIG. 6A) One intravenous injection of KLF2 mRNA encapsulated, VCAM1-targeting lipid nanoparticles significantly increases KLF2 mRNA in the mouse lung subjected to infection of SARS-COV-2 viruses compared to those in H1N1-infected mice administered with PBS. (FIG. 6B) SARS-COV-2 virus-induced lung inflammation is significantly reduced by intravenous injections of KLF2 mRNA encapsulated, VCAM1-targeting lipid nanoparticles. SARS-COV-2 infection significantly increases mouse lung inflammation, demonstrated by increased expression of CCL2 and CCL4. Intravenous injections of KLF2 mRNA encapsulated, VCAM1-targeting lipid nanoparticles significantly reduce the expression of CCL2 and CCL4 in the SARS-COV-2-infected mouse lung.

FIG. 7 shows that N1-methylpseudouridine (m1ψ) substitution of uridine enhanced the protein output of mScarlet mRNA in pulmonary microvascular endothelial cells (PMVECs). (n=5, representative images shown).

FIG. 8 shows the increased half-life of Cy5-labeled mRNA (~1 kb) encapsulated in the VCAM1-targeting lipid nanoparticle in mouse circulation. (n=3). Data are mean±SEM.

FIG. 9 shows that KLF2 mRNA-encapsulated, VCAM1-targeting lipid nanoparticles effectively deliver functional KLF2 mRNA to the inflamed mouse lung subjected to influenza H1N1 viruses, leading to the upregulation of eNOS and RAPGEF3 that are KLF2 downstream targets.

FIGS. 10A-10B show that KLF2 mRNA-encapsulated, VCAM1-targeting lipid nanoparticles significantly reduce lung injury induced by influenza H1N1 viruses in vivo. (FIG. 10A) Intravenous injections of KLF2 mRNA encapsulated, VCAM1-targeting lipid nanoparticles significantly reduce the total cell counts in the bronchoalveolar lavage (BAL), a major surrogate of lung edema and injury. (FIG. 10B) Intravenous injections of KLF2 mRNA encapsulated, VCAM1-targeting lipid nanoparticles significantly reduce H1N1-induced lung injury by reducing the total protein in the bronchoalveolar lavage (BAL).

FIG. 11 shows that KLF2 mRNA-encapsulated, VCAM1-targeting lipid nanoparticles significantly reduce lung pathology induced by influenza H1N1 viruses in vivo. Mouse lung infection of influenza H1N1 viruses leads to lung injury, detected in H&E stained histological lung sections and quantified by the lung histological (pathological) score. Intravenous injections of human KLF2 mRNA encapsulated, VCAM1-targeting lipid nanoparticles significantly reduce H1N1-induced lung injury quantified by the histological (pathological) score in H&E stained lung sections. In contrast, intravenous injections of control (non-functional) mRNA encapsulated, VCAM1-targeting lipid nanoparticles have no effect on the H1N1-induced lung injury quantified by the histological (pathological) score.

FIG. 12A-12C OAS1 mRNA-encapsulated, epithelium-targeting lipid nanoparticles effectively deliver functional OAS1 mRNA to mouse lung. FIG. 12A demonstrates that intratracheal installations of epithelium-targeting lipid nanoparticles effectively delivered functional mSCarlet mRNA to mouse ARDS lung induced by influenza A virus. The lipid nanoparticle was engineered to display the peptide CTSGTHPRC (SEQ ID NO: 8) which binds epithelium, a sequence described by Morris et al. (J. Control. 2011, 151 (1), 83-94). FIG. 12B shows that intratracheal installations of OSA1 mRNA-encapsulated epithelium-targeting lipid nanoparticles significantly reduced the viral counts of influenza A virus in the mouse lung, quantified by the viral gene NS1. FIG. 12C demonstrates that intratracheal installations of OSA1 mRNA-encapsulated epithelium-targeting lipid nanoparticles significantly reduced the mouse lung injury induced by influenza A virus, quantified by the total cell counts in the BAL. Data are means+SD.

### DETAILED DESCRIPTION

Provided herein are compositions and methods for treating treating lung disorders, including, for example, Acute Respiratory Distress Syndrome (ARDS), Ventilator-Induced Lung Injury (VILI), Acute lung injury (ALI), and other acute and chronic lung disorders.

As used herein, the term “lung disorder” refers to disorders, diseases, and/or damage to the lungs of an individual. As used herein, the term “lung disease” can also be described as a “lung disorder” or “lung injury.” For example, Acute Respiratory Distress Syndrome (ARDS), Ventilator-Induced Lung Injury (VILI), Acute lung injury (ALI), and other acute and chronic lung disorders.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

In some embodiments, percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

As used herein, the terms “therapeutically effective amount” or “effective amount” refer to that amount of a therapeutic agent, such as a lipid nanoparticle comprising an RNA molecule encoding KLF2, which when administered to a subject, is sufficient to effect treatment (e.g., improve symptoms) for a disease or disorder described herein, such as, for example, Acute Respiratory Distress Syndrome (ARDS), Ventilator-Induced Lung Injury (VILI), Acute lung injury (ALI), and other acute and chronic lung disorders. The amount of a compound which constitutes a “therapeutically effective amount” or “effective amount” can vary depending on the compound, the disorder and its severity, and the age, weight, sex, and genetic background of the subject to be treated, but can be determined by one of ordinary skill in the art.

As used herein, the terms “treating” or “treatment” refer to the treatment of a disease or disorder described herein, in a subject, preferably a human, and includes inhibiting, relieving, ameliorating, or slowing progression of the disease or disorder or one or more symptoms of the disease or disorder.

As used herein, the term “subject” refers to a warm blooded animal such as a mammal, preferably a human, which is afflicted with, or has the potential to be afflicted with one or more diseases and disorders described herein.

As used herein, the term “pharmaceutical composition” refers to a composition that includes one or more therapeutic agents disclosed herein, such as an RNA molecule encoding KLF2, a pharmaceutically acceptable carrier, a solvent, an adjuvant, and/or a diluent, or any combination thereof.

In view of the present disclosure, the methods and compositions described herein can be configured by the person of ordinary skill in the art to meet the desired need. In general, the disclosed materials and methods provide improvements in treating lung disorders as described herein.

Disclosed herein is a targeted approach to restore KLF2 mRNA in inflamed endothelium to treat ALI/ARDS/VILI in a subject. In some embodiments, the ALI/ARDS/VILI can be induced by influenza or SARS-COV-2 viruses. The engineered targeted lipid nanoparticles successfully deliver KLF2 mRNA to inflamed endothelium via a targeting peptide against Vascular Cell Adhesion Molecule 1 (VCAM-1). Data as presented herein demonstrated that this targeting strategy toward inflamed endothelium reduces ALI/ARDS/VILI induced by SARS-COV-2 in a mouse model. Furthermore, the targeted lipid nanoparticles were tested in treating ALI/ARDS/VILI induced by influenza or SARS-COV-2 viruses in mice. N1-methylpseudouridine (m1!′) substitution of the uridine, an RNA modification associated with enhanced protein expression and reduced cellular immunogenicity in COVID-19 mRNA vaccine, was incorporated in the KLF2 mRNA.

The significance of the present disclosure includes at least two aspects. First, it provides novel nanomedicine approaches to treat lung disorders with unmet medical need. Second, it integrates targeted nanomedicine and RNA therapeutics to create a new avenue for the treatment of various lung diseases including Acute Respiratory Distress Syndrome (ARDS), Ventilator-Induced Lung Injury (VILI), Acute lung injury (ALI), and other acute and chronic lung disorders. This disclosure further provides, in part, a peptide-targeted lipid nanoparticles to deliver therapeutic mRNA to endothelial cells, enhancing KLF2 mRNA expression and reducing viral titers compared to a control at the site of the inflamed endothelial cells.

### Compositions

In some embodiments, pharmaceutical compositions contemplated herein include a therapeutically effective amount of a targeted lipid nanoparticle including one or more inhibitors of endothelial inflammation, such as, for example, an RNA molecule encoding KLF2. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

In some embodiments, pharmaceutical compositions contemplated herein include one or more lipid nanoparticles that carry one or more mRNA enhancers, such as an RNA molecule encoding KLF2, for example, inside the lipid nanoparticle, attached to an external surface of the lipid nanoparticle, or both. In some embodiments, the lipid nanoparticles include one or more targeting moeities attached thereto to enable targeted delivery of the lipid nanoparticle to a desired location and/or cell type. For example, the targeting moeity can target the lipid nanoparticle to a site of endothelial inflammation associated with a lung disease or disorder or wound.

Any therapeutic mRNA is contemplated herein. For example, contemplated mRNAs include KLF2 mRNA or anti-viral genes OAS⅓.

Such compositions optionally include secondary therapeutic agents (possibly also carried on or in contemplated lipid nanoparticles).

In some embodiments, the mRNA molecule encoding KLF2 and/or OAS1, of the present disclosure can be administered through a variety of routes and in various compositions. For example, pharmaceutical compositions comprising lipid nanoparticles containing the RNA molecule encoding KLF2 and/or OAS1, can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, lipid nanoparticles containing one or more an RNA molecules encoding KLF2 and/or OAS1, are administered orally, and the subject is dosed on a daily basis.

The therapeutic agents (also referred to as “compounds” herein) described herein (e.g., lipid nanoparticle, VCAM-1 targeting molecule, mRNA), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC50 of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of an RNA molecule encoding KLF2 can be in the range of from about 0.0001 mg/kg/day, about 0.001 mg/kg/day, or about 0.01 mg/kg/day, or about 0.1 mg/kg/day, or about 1.0 mg/kg/day, or about 10 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 mL of a pharmaceutical composition comprising lipid nanoparticles having a concentration of mRNA encoding KLF2 at about 0.00001, 0001, 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 1000 mM in a pharmaceutically acceptable carrier.

### Lipid Nanoparticles

Lipid nanoparticles (LNP) are colloidal dispersions that are composed of one or more lipid-bilayers that surround an aqueous core. The ability of lipid nanoparticles to encapusulate lipophilic and hydrophlic drugs have allowed these vesicles to be useful drug delivery systsems. Important physicochemical properties of lipid nanoparticles such as the hydrodynamic diameter or particle size, surface charge (typically measured as zeta-potential), lipid-packing, bilayer lamellarity, encapsulation efficiency, drug encapsulation, molecular loading and external modifications (such as polymer coatings and targeting moiety incorporation) are necessary to accurately control and measure to properly manufacture a pharmaceutical drug product. Lipid nanoparticles can be formed to have a hydrodynamic diameter (in nanometers [d.nm]) ranging from approximately 30 d.nm to over 500 d.nm. For lipid nanoparticles that are less than 500 d.nm, these particles exhibit Brownian motion and remain as a colloidal dispersion since the thermal motion of the particles overcome gravitational forces that would otherwise increase the likelihood of sedimentation.

Contemplated lipid nanoparticles for use herein include, for example, the lipid nanoparticle comprises a polyamidoamine (PAMAM) dendrimer (G0-C14) (for example, a lipid grafted polyamidoamine (PAMAM) dendrimer (G0-C₁₄)), cholesterol, polyethylene glycol 2000 (PEG), 1,2-Distearoyl-sn-glycero-3-phosphoethanolamine-Poly(ethylene glycol) (DSPE-PEG), dioleoylphospha-tidylethanolamine (DOPE), and KLF2 mRNA encapsulated by the lipid nanoparticle. In some embodiments, the PEG domain comprises PEG having an average molecular weight of about 1,000 to about 100,000 Daltons. In some embodiments, the VCAM-1 targeting molecule comprises a peptide comprising the amino acid sequence VHPKQHR (SEQ ID NO: 1) or DITWDQLWDLMK (SEQ ID NO: 3). In some embodiments, the VCAM-1 targeted lipid nanoparticle comprises DSPE-PEG-VHPKQHR (SEQ ID NO:6) or DSPE-PEG-DITWDQLWDLMK (SEQ ID NO:7). In some embodiments, the RNA molecule encodes KLF2 mRNA. In some embodiments, the KLF2 mRNA is a m1Ψ-substituted KLF2 mRNA. In some embodiments, the KLF2 mRNA comprises a concentration of about 0.01 μM to about 100 μM. In some embodiments, the KLF2 mRNA comprises a concentration of about 2 μM. In some embodiments, the lipid nanoparticle comprises a molar ratio of the lipid grafted polyamidoamine (PAMAM) dendrimer (G0-C₁₄) to DSPE-PEG is about 2:1 to about 15:1; cholesterol to DSPE-PEG is about 15:1 to about 40:1; and DOPE to DSPE-PEG is about 15:1 to about 30:1. In some embodiments, the lipid nanoparticlecomprises molar ratios of lipid grafted PAMAM G0-C14:DOPE:Cholesterol:DSPE-PEG of 5:15:25:1.

Additional micelles are contemplated for use herein, such as those disclosed in International Application No. PCT/US2006/020760, Vieregg et al. (J. Am. Chem. Soc. 2018, 140, 1632-1638), Lueckheide et al. (Nano Lett. 2018, 18, 7111-7117), and Marras et al. (Polymers 2019, 11,83), each of which is incorporated by reference in its entirety.

### Targeting Molecules

The present disclosure contemplates use of targeting molecules (or targeting moieties) with the lipid nanoparticles disclosed herein for targeted delivery of therapeutic compositions, such as an RNA molecule encoding KLF2, or for incorporation into pharmaceutical compositions as described herein. Targeting molecules can be VCAM1-targeting molecules. Targeting molecules can include peptides such as VHPKQHR (SEQ ID NO: 1), which was identified via phage display and allows for targeting of lung endothelial cells through VCAM-1. Peptide targeting molecules further include the amino acid sequence DITWDQLWDLMK (SEQ ID NO: 3), which allows for the targeting E-selectin. E-selectin is a cell adhesion molecule expressed only on cytokine-activated endothelial cells.

The expression of vascular cell adhesion molecule 1 (VCAM1) is low in healthy endothelium but increases in inflamed endothelial cells (ECs). To achieve effective targeting to VCAM1-expressing ECs, the lipid nanoparticles are functionalized with a VCAM1 binding peptide that has been shown to facilitate VCAM1-mediated intracellular internalization of nano-materials in endothelium in vitro and in vivo. In some embodiments, contemplated targeting peptides are positioned at the periphery of the corona of the lipid nanoparticles.

In one embodiment, a contemplated targeted lipid nanoparticle containing an RNA molecule encoding KLF2 (2 μM) is DSPE-PEG-VHPKQHR (SEQ ID NO:6) or DSPE-PEG-DITWDQLWDLMK (SEQ ID NO:7).

In some embodiments, contemplated lipid nanoparticles containing an RNA molecule encoding KLF2 exhibit a polydispersity of about 0.1 to about 0.3.

In some embodiments, contemplated lipid nanoparticles containing an RNA molecule encoding KLF2 contained with the core exhibit a spherical shape and have a diameter (in nanometers, nm) of about 10, 11, 12, 13, 14, 15, 16, 17, 18, 19, 20, 21, 22, 23, 24, 25, 30, 35, 40, 45, or 50 nm. In some embodiments, contemplated lipid nanoparticles exhibit a spherical shape and have a diameter (in nanometers, nm) of about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, about 70, about 75, about 80, about 85, about 90, about 95, about 100, about 105, about 110, about 115, about 120, or about 125 nm. In some embodiments, the nanoparticles have a radius of about 40 nm to about 80 nm. In some embodiments, the nanoparticles have a radius of about 20 nm to about 100 nm.

The lipid nanoparticle delivery system of the present disclosure possesses multiple advantages compared with other lipid nanoparticle-based platforms. The first advantage is higher stability: the therapeutically active components (e.g., an RNA molecule encoding KLF2, or other nucleic acid based therapeutics) can be encapsulated in the inner core of the nanoparticle. Through this approach, first, the degradation of nucleotides of the RNA molecule encoding KLF2 by serum nucleases is prevented; second, the lipid nanoparticles are capable of escaping renal clearance; and third, the immunogenic responses are avoided. The second advantage is higher safety: cell-targeting peptides (e.g., those targeting to Vascular Cell Adhesion Molecule 1 (VCAM-1)) are covalently conjugated on the periphery of the lipid nanoparticles, which significantly reduces cytotoxicity and increases circulation time by circumventing nonspecific interaction with serum components. The third advantage is higher specificity: with the defined chemical structures of the targeting peptides, the lipid nanoparticles are able to bind specific receptors and penetrate targeted cells. The final advantage is higher scalability. This approach does not require chemical modifications on nucleotides for conjugation, nor does it need to engineer hard-to-reproduce lipid nanoparticles. The synthesis of the core components in the lipid nanoparticles is highly automated. In addition, the targeting peptides are easily changeable to target different receptors.

Further, as shown herein, the use of targeted lipid nanoparticles permits use of a lower amount of a therapeutic agent for the treatment of a lung disorder or wound due to the specific targeting of the therapeutic agent to the site of the lung disorder or wound. In this way, use of targeted lipid nanoparticles can significantly lower the dosage of a therapeutic agent required to treat a lung disorder or wound, which can significantly reduce costs associated with the treatment. For example, a therapeutically effective amount of a therapeutic agent to be delivered by a targeted lipid nanoparticle can be at least about 10, 20, 30, 40, or 50% lower than the therapeutically effective amount of the naked (non-targeted) therapeutic agent.

Lipid nanoparticles are colloidal dispersions that are composed of one or more lipid-bilayers that surround an aqueous core. The ability of lipid nanoparticles to encapusulate lipophilic and hydrophlic drugs have allowed these vesicles to be useful drug delivery systsems. Important physicochemical properties of lipid nanoparticles such as the hydrodynamic diameter or particle size, surface charge (typically measured as zeta-potential), lipid-packing, bilayer lamellarity, encapsulation efficiency, drug encapsulation, molecular loading and external modifications (such as polymer coatings and targeting moiety incorporation) are necessary to accurately control and measure to properly manufacture a pharmaceutical drug product. Lipid nanoparticles can be formed to have a hydrodynamic diameter (in nanometers [d.nm]) ranging from approximately 30 d.nm to over 500 d.nm. For lipid nanoparticles that are less than 500 d.nm, these particles exhibit Brownian motion and remain as a colloidal dispersion since the thermal motion of the particles overcome gravitational forces that would otherwise increase the likelihood of sedimentation.

### Methods

In some embodiments, methods of treating and/or preventing a lung disorder in a subject in need thereof include administering to the subject a therapeutically effective amount of one or more KLF2 enhancers (e.g. one or more molecules that results in the overexpression of and/or increased activity of KLF2), and/or a therapeutically effective amount of one or more OAS1 enhancers (e.g. one or more molecules that results in the overexpression of and/or increased activity of OAS1). Treatable and/or preventable lung disorders can include Acute Respiratory Distress Syndrome (ARDS), Ventilator-Induced Lung Injury (VILI), Acute lung injury (ALI), and other acute and chronic lung disorders.

In some embodiments, therapeutic methods contemplated herein can also treat and/or prevent complications associated with or promote endothelial wound healing (e.g., caused by lung injury) by administering to the subject a therapeutically effective amount of an RNA molecule encoding KLF2 or OAS1. For example, treatment and/or prevention of complications associated with endothelial wound healing is associated with the reduction of inflammation at the site of the wound and the stimulation of endothelial growth.

In some embodiments, therapeutic methods contemplated herein can also accelerate endothelial growth to treat wound healing (e.g., caused by lung injury) by administering to the subject a therapeutically effective amount of an RNA molecule encoding KLF2 or OAS1.

Acute and chronic lung diseases, contemplated by the present disclosure, are major causes of mortality and morbidity in the US. Acute Respiratory Distress Syndrome (ARDS) is a severe form of lung injury, in which the alveolar-vascular barrier breaks down, causing vascular exudate to flood the breathing units of the lung, resulting in hypoxia and the need for advanced respiratory support. ARDS is the major cause of death in critically ill patients suffering from influenza or SARS-COV-2 viral infections.

The present disclosure contemplates a variety of methods of administering the therapeutic agents, targeting molecules, and lipid nanoparticles disclosed herein, including local, oral, nasal, rectal, intravaginal, topical, subcutaneous, intradermal, intramuscular (IM), intravenous (IV), intrathecal (IT), intracerebral, epidural, or intracranial administration. Local, in situ administration of these compositions is contemplated.

The present disclosure contemplates methods that result in a variety of indications of improvement for respiratory distress syndrome/acute respiratory distress syndrome (ARDS) induced by influenza or SARS-COV-2 viruses in the patient.

The present disclosure contemplates use of the disclosed methods in conjunction with other treatments for respiratory distress syndrome/acute respiratory distress syndrome (ARDS) induced by influenza or SARS-COV-2 viruses in the patient.

The present disclosure contemplates methods that result in a variety of indications of improvement for respiratory distress syndrome/acute respiratory distress syndrome (ARDS) induced by bleomycin, influenza, or SARS-COV-2 viruses.

The present disclosure contemplates use of the disclosed methods in conjunction with other treatments for respiratory distress syndrome/acute respiratory distress syndrome (ARDS) induced by bleomycin, influenza, or SARS-COV-2 viruses.

### EXAMPLES

The Examples that follow are illustrative of specific embodiments of the disclosure, and various uses thereof. They are set forth for explanatory purposes only and should not be construed as limiting the scope of the disclosure in any way.

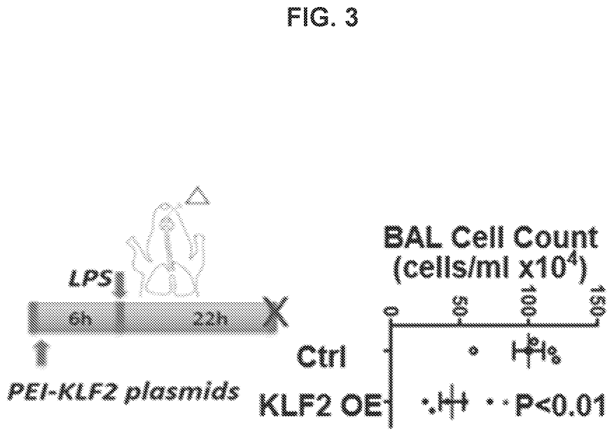

The present disclosure leverages unique, cell type-specific lung biology that was discovered to target specific cell types employing innovative lipid nanoparticles for gene delivery. Acute Respiratory Distress Syndrome (ARDS), caused by endothelial dysfunction and uncontrolled cytokine storm, is the major cause of death in critically ill patients infected with influenza or SARS-COV-2 viruses. ARDS treatments do not directly target dysfunctional endothelium, underscoring an unmet medical need heightened by the COVID-19 pandemic. KLF2 overexpression, using non-targeting PEI nanoparticles and KLF2 plasmids, before intratracheal delivery of LPS significantly reduces ARDS in mice (FIG. 3) demonstrated by Huang et al. (Am. J. Respir. Crit. 2017, 195 (5), 639-651). However, it remains unknown whether endothelial KLF2 restoration treats ARDS after the influenza or SARS-COV-2 viral infections.

### Materials and Methods

The research approach described here employed complementary in vitro, in vivo (mouse) and ex vivo (human) investigations of lung injury. Human lung experiments included at least 12 biological replicates (distinct human donors); data reported reflect these biological replicates and not technical replicates. Translational animal experiments were conducted in accordance with the ARRIVE guidelines. Both male and female mice were blindly randomized to experimental or control groups, minimizing subjective bias and controlling for sex effects of disease (where appropriate-fibrosis is regulated by mouse sex). Rigorous contemporaneous controls were used. All analyses were performed by observers blinded to treatment allocation. All assays were performed in a blinded manner in triplicate. Statistical outliers were explicitly reported. In vivo animal studies include the experimental treatment with lipid nanoparticles to determine effectiveness for reducing ALI/ARDS. Using Stata software version 6, a power of analysis calculation from previous studies, an estimated minimum of 25 mice per condition were required to reach significance for analyses. A biostatistician was involved with all aspects of study design, data collection, and analysis.

### Example 1. Klf2 is Significantly Reduced in Mouse Lung Treated with Lipopolysaccharide (Lps), Influenza a Virus or Sars-Cov-2 Virus, and in Rat Ali/Ards Lung Subjected to High-Tidal Ventilation

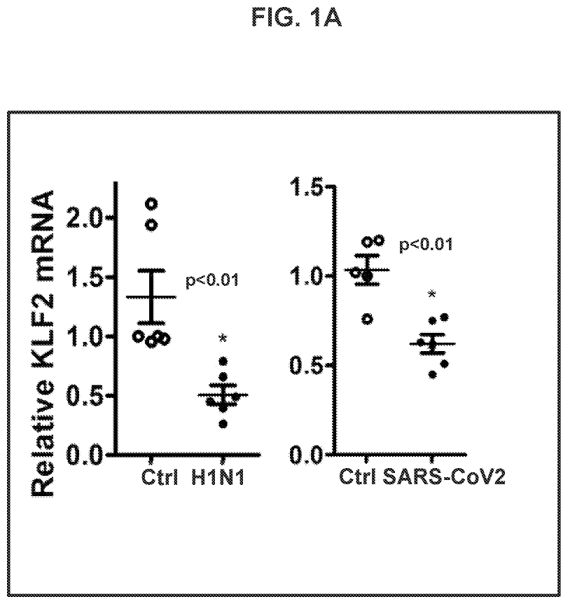

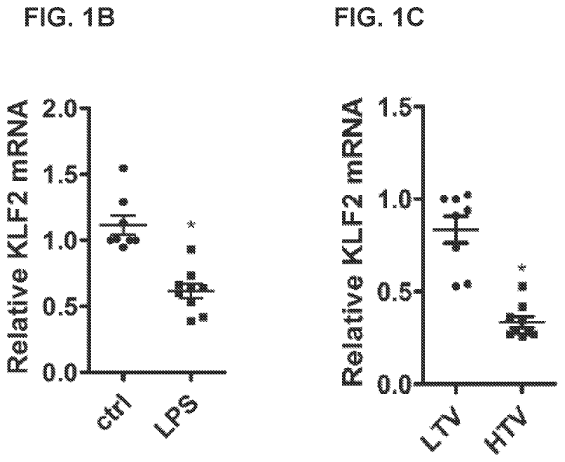

Krüppel-like factor 2 (KLF2) is a lung-enriched transcription factor that promotes endothelial heath that prevents vascular permeability and induces anti-viral responses and is significantly reduced in animal models of ALI/ARDS. Intratracheal instillation either with pandemic influenza A H1N1 (A/WSN/33 [H1N1]) virus FIG. 1) or lipopolysaccharide significantly reduces KLF2 in the B6 mouse lung, which causatively drives pulmonary endothelial dysfunction and ALI/ARDS described in Huang et al. (Am. J. Respir. Crit. 2017, 195 (5), 639-651). As shown in FIG. 1, intratracheal instillation of SARS-COV-2 also markedly reduces KLF2 mRNA in lung of K18-hACE2 mice (a strain prone to SARS-COV-2 infection).

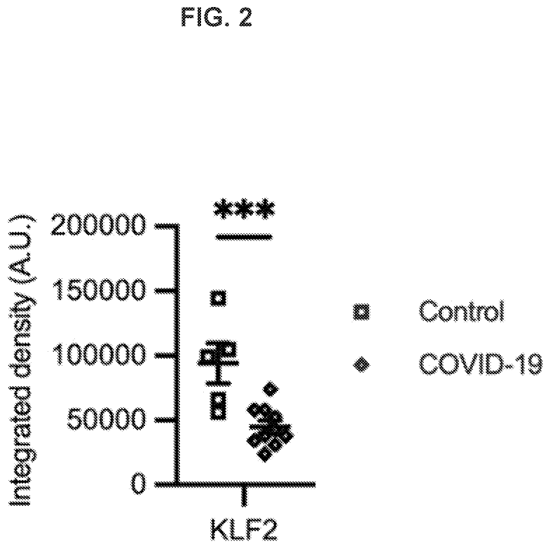

KLF2 is significantly reduced in human lung infected with SARS-COV-2: Recent published studies, such as Wu et al. (Am. J. Respir., 2021, 65 (2), 222-226), in lung autopsy samples from COVID-19 patients demonstrated that KLF2, enriched in vascular endothelium, is significantly reduced in SARS-COV-2-infected lungs when compared to healthy human lung tissues (FIG. 2). Moreover, results demonstrated that KLF2 is a major transcriptional activator of OAS1 and OAS3 (2′-5′-oligoadenylate synthetase 1 and 3), two key antiviral genes against virus infection which were recently implicated in COVID-19 severity by a genome-wide association study from Pairo-Castineira et al. (Nature, 2021, 591 (7848), 92-98).

Prophylactic KLF2 overexpression reduces LPS-induced ALI/ARDS in mice: ARDS is currently underserved by the nanomedicine research community. Recent results demonstrated a proof-of-concept employing nanomedicine to significantly reduce ALI/ARDS in mice shown by Huang et al. (Am. J. Respir. Crit. 2017, 195 (5), 639-651). Non-targeting polyethylenimine (PEI) nanoparticles were used to overexpress KLF2 in mouse lung, which reduced LPS-induced ARDS, demonstrated by decreased total cells (see FIG. 3) and total protein (data not shown) in the bronchoalveolar lavage (BAL) fluid. There are three major challenges to bring this to clinical settings to treat critically ill influenza and COVID-19 patients. First, prophylactic effect was tested by administering the PEI nanoparticles 6 hours before the ARDS was induced (see FIG. 3). Second, the effect of KLF2 restoration in treating ARDS induced by influenza or SARS-COV-2 viruses remains unknown. Third, the PEI nanoparticle has no endothelium-targeting capacity and could lead to KLF2 overexpression in unwanted tissues.

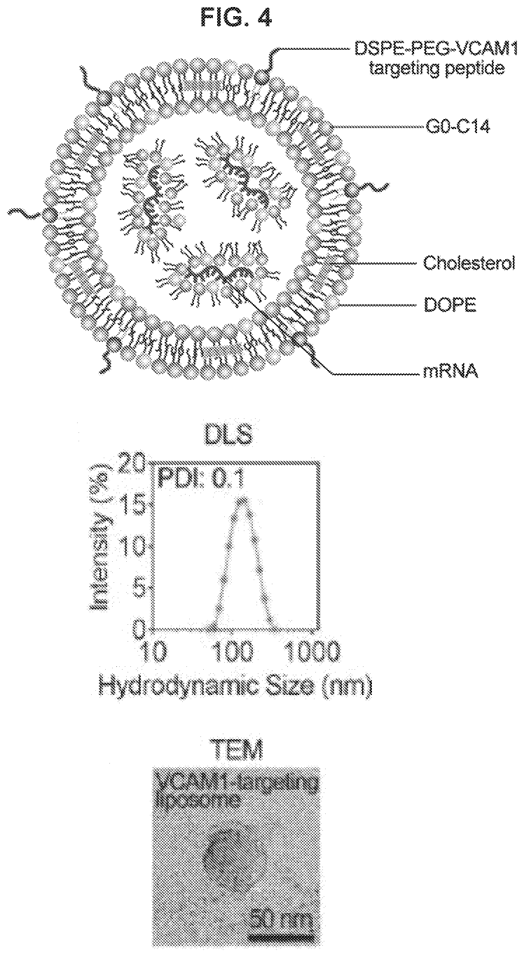

These limitations can be addressed comprehensively and rapidly by testing the therapeutic effects of targeted nanomedicine in treating influenza and SARS-COV-2-induced ARDS in vivo. Therefore, an optimized lipid nanoparticle-based, vascular cell adhesion molecule-1 (VCAM1)-targeting lipid nanoparticle was engineered to deliver KLF2 mRNA to inflamed vascular endothelium (FIGS. 4-9). In previous studies, targeted nanoparticles encapsulating diagnostic or therapeutic agents were formulated, including a polyelectrolyte complex micelle that preferentially delivers microRNA inhibitors to inflamed endothelium by targeting of VCAM-1 using the peptide VHPKQHR (SEQ ID NO: 1) as described by Zhou et al. (Proc. Natl. Acad. Sci. U.S.A, 2021, 118 (50), e2114842118). Lipid nanoparticles have been used in FDA-approved lipid nanoparticles for human use. Therefore, the therapeutic effectiveness of this VCAM1-targeting lipid nanoparticle in treating influenza or COVID-19-induced ARDS was determined in a mouse model.

Formulation, optimization, and characterization of VCAM1-targeting, KLF2 mRNA-encapsulated lipid nanoparticles:For the formulation and optimization of KLF2 mRNA-encapsulated lipid nanoparticles, the formulation of a lipid nanoparticle was engineered, screened and optimized to achieve effective cytosolic delivery of functional KLF2 mRNA. Self-assembled lipid nanoparticles were formed from a mixture of lipid grafted polyamidoamine (PAMAM) dendrimer (G0-C14), cholesterol, polyethylene glycol 2000 (PEG), 1,2-Distearoyl-sn-glycero-3-phosphoethanolamine-Poly(ethylene glycol) (DSPE-PEG), dioleoylphospha-tidylethanolamine (DOPE), and KLF2 mRNA. A library of non-targeting lipid nanoparticles was first optimized and screened for effective endosome/lysosome escape of KLF2 mRNA that can be functionally translated. A library of 64 lipid nanoparticles with distinct molar ratios of the building components was established (Table 1) and tested for their capacity to deliver functional KLF2 mRNAs to cultured human pulmonary microvascular endothelial cells (PMVECs).

TABLE 1
Formulations of the lipid nanoparticle library.
Components (molar ratios)

| G0-C14 | DOPE | Cholesterol | DSPE-PEG |
|---|---|---|---|
| 2, 5, 10, or 15 | 15, 20, 25, or 30 | 15, 20, 30, or 40 | 1 |

Previous studies demonstrated that in PMVEC, Rap guanine nucleotide exchange factor 3 (RAPGEF3, a key molecule to maintain the endothelial monolayer integrity) is one of the most responsive direct downstream targets transcriptionally up-regulated by KLF2 shown by Huang et al. (Am. J. Respir. Crit. 2017, 195 (5), 639-651). Therefore, RAPGEF3 expression, a surrogate of functional KLF2 mRNA transfection was detected in lipid nanoparticle-treated (200 ng RNA in a 24 well, 24 hrs incubation) PMVEC. Results identified that the lipid nanoparticle with this molar ratio (G0-C14: 5, DOPE: 15; Cholesterol: 25, DSPE-PEG: 1) resulted in the most up-regulation (>5 fold) of RAPGEF3 and did not induce immune responses (IL-6 and CCL2, data not shown) in PMVECs.

Functionalization and characterization of VCAM1-targeting, KLF2 mRNA-encapsulated: To understand the functionalization and characterization of VCAM1-targeting, KLF2 mRNA-encapsulated lipid nanoparticles (comprising G0-C14: 5, DOPE: 15; Cholesterol: 25, DSPE-PEG: 1) was prioritized for functionalization to target inflamed vascular endothelium by displaying the VCAM1-binding peptide (VHPKQHR (SEQ ID NO: 1)) on the surface. The VCAM1-targeting peptide was incorporated to the DSPE-PEG molecule (DSPE-PEG-VHPKQHR (SEQ ID NO:6) in FIG. 4). VHPKQHR (SEQ ID NO: 1) was identified by phage display to specifically bind VCAM-1 in activated endothelium and moreover, was shown to facilitate VCAM1-mediated intracellular internalization of nano-materials in endothelium in vitro and in vivo. In a recent study, the VCAM1-targeting strategy was employed to deliver small RNA (miR-92a inhibitor)-encapsulated polyelectrolyte complex micelles to lessen vascular diseases (atherosclerosis and stenosis) induced by chronic inflammation demonstrated in Zhou et al. (Proc. Natl. Acad. Sci. U.S.A, 2021, 118 (50), e2114842118). Transmission electron microscopy (TEM) images showed monodisperse VCAM1-targeting lipid nanoparticles with a diameter of ~70 nm in dry condition and a median hydrodynamic diameter of ~120 nm in deionized (DI) water (FIG. 4).

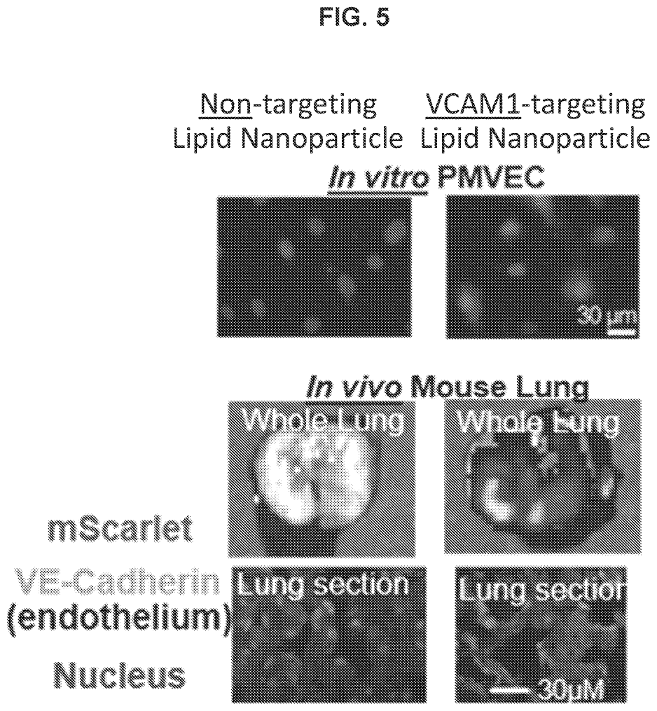

Results demonstrated that in inflamed PMVECs in which VCAM-1 expression is significantly induced by lipopolysaccharides (LPS), VCAM1-targeting significantly enhances VCAM1-targeting lipid nanoparticles to deliver the functional mRNA of mScarlet (a bright monomeric red fluorescent protein) when the lipid nanoparticle treatment is 30 minutes, compared to the non-targeting lipid nanoparticle (FIG. 5). Moreover, the in vivo studies (FIG. 5) demonstrated that these VCAM1-targeting lipid nanoparticles effectively delivered functional mScarlet mRNA to inflamed mouse lungs subjected to LPS while non-targeting lipid nanoparticles showed limited potency of functional mRNA delivery. The lipid nanoparticle is engineered to display a non-functional scrambled (QAHPHVD (SEQ ID NO: 2)) VCAM1-targeting peptide to serve as an additional control.

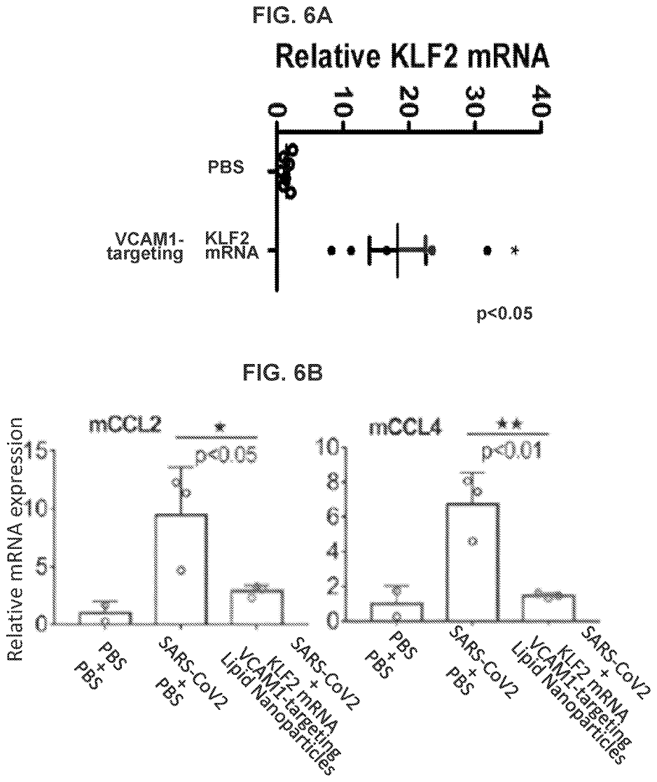

VCAM1-targeting lipid nanoparticles effectively deliver KLF2 to inflamed lung endothelium and significantly reduce mouse ARDS induced by SARS-COV-2: The therapeutic effectiveness of the VCAM1-targeting, KLF2 mRNA-encapsulated lipid nanoparticles was tested in reducing SARS-COV-2 viral load in mice. The experiments were conducted in the BSL-3 SARS core in the Argonne National Laboratory operated by the University of Chicago. The SARS-COV-2 was propagated and the K18-hACE2 transgenic mouse line (from Jackson Lab) in which human angiotensin-converting enzyme 2 (hACE2, a functional human receptor for the coronaviruses) was knocked-in was secured. K18-hACE2 mice were intratracheally instilled either with SARS-COV-2 (10⁵ TCID₅₀/50 μl) or control PBS (50 μL). 24 hours after intratracheal installation, mice were intravenously injected with VCAM1-targeting KLF2 lipid nanoparticles (1 mg mRNA/kg body weight, total 100 μL). Two days after the lipid nanoparticle injections, lungs were collected, showing that VCAM1-targeting lipid nanoparticles effectively reduced lung injury (FIG. 6).

### Example 2: Determining the therapeutic effectiveness of VCAM1-targeting

lipid nanoparticles for treating ALI/ARDS induced by influenza A virus (A/WSN/33 [H1N1]) or SARS-COV-2.

The main objective is to determine the therapeutic effects of VCAM1-targeting, KLF2 mRNA-encapsulated lipid nanoparticles in reducing ARDS induced by SARS-COV-2 or H1N1 influenza viruses. FIG. 1 demonstrates that influenza or SARS-COV-2 infections significantly reduced KLF2 mRNA expression in the lungs. Moreover, results show that VCAM1-targeting lipid nanoparticles effectively delivered functional KLF2 mRNA to inflamed lung tissue in mice (FIG. 5). Furthermore, the results demonstrated that KLF2 mRNA-encapsulated, VCAM1-targeting lipid nanoparticles significantly reduced lung inflammation induced by SARS-COV-2 viruses, demonstrated by decreased expression of CCL2 and CCL4 (FIG. 6). This example systematically determined the lung functions and pathophysiology as a function of viral infections and NP treatment. Modified nucleobase N1-methylpseudouridine (m1′) substitution of the uridine (used in the COVID-19 vaccines), an RNA modification associated with enhanced protein expression and reduced immunogenicity in COVID-19 mRNA vaccine, was incorporated in the KLF2 mRNA. m1P-substituted KLF2 mRNAs were used in this Example, and enhanced the therapeutic effectiveness treating mouse ALI/ARDS when compared to the preliminary studies using regular KLF2 mRNA molecules. Experimental design: Mouse ARDS was induced by intratracheal delivery of H1N1 influenza virus. Intravenous injections of VCAM1-targeting lipid nanoparticles encapsulating control mutant mRNA or functional KLF2 mRNA were conducted on day 1 and day 4 after the H1N1 influenza virus inoculation. Mice were sacrificed on day 6.

VCAM1-targeting, KLF2 mRNA-encapsulated lipid nanoparticle: Briefly, 30 μL ethanol containing 25 μg lipid-PAMAM (50 mg/mL in ethanol), 25 μg cholesterol (20 mg/mL in ethanol), 28 μg DOPE (20 mg/mL in ethanol) and 5 μg DSPE-PEG-VHPKQHR (SEQ ID NO: 6; 1 mg/mL in ethanol) were added to 90 L DNase/RNase-free DI water containing 2 μg KLF2 mRNA (0.43 μg/μL in DI water) to form lipid nanoparticles (FIG. 4). N1-Methylpseudouridine (m1ψ)-substituted KLF2 mRNA was used. The size and zeta potential of lipid nanoparticles were determined by dynamic light scattering (DLS) and cryogenic transmission electron microscopy (Cryo-TEM).

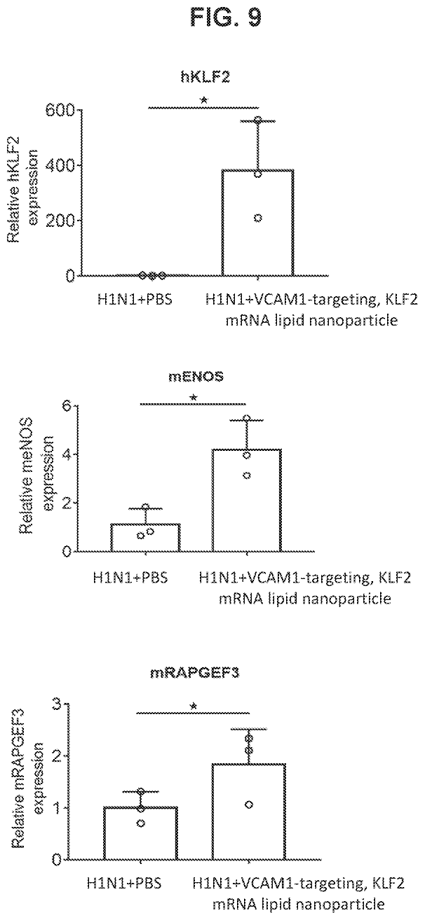

Application of VCAM1-targeting lipid nanoparticles in treating lung injury in mice induced by influenza A virus (A/WSN/33 [HIN1]): A/WSN/33 is an H1N1 influenza virus strain that infects human and mouse lungs, causing ARDS symptoms. Studies using A/WSN/33 [H1N1] can be conducted in a BSL-2 lab. A previous study showed that intratracheally instilled A/WSN/33 [H1N1](500 pfu/mouse) resulted in acute lung injury as manifested by elevated cell counts and proteins in bronchoalveolar lavage (BAL) fluid. Detailed protocols of intratracheally instilled A/WSN/33 [H1N1] in mice have been described in a previous study. Briefly, C57BL/6J mice were anesthetized and then intratracheally instilled with influenza A virus (A/WSN/33 [H1N1], 500 pfu/mouse, day 0). After intratracheal installation, mice will be intravenously injected with VCAM1-targeting KLF2 lipid nanoparticles (1 mg mRNA/kg body weight, in 100 μl solution) on day 1 and day 4. A total of 4 groups (all in in 6-8 weeks old mice, both male and female) were employed in this study: 1) Intratracheal PBS (control), 2) H1N1 virus instilled/PBS tail vein injection, 3) H1N1 virus instilled/VCAM-1 targeting, control mRNA (KLF2 mutant mRNA that cannot be translated) lipid nanoparticles, and 4) H1N1 virus instilled/VCAM-1 targeting, KLF2 mRNA lipid nanoparticles. 48 hours after tail vein injections, animals were sacrificed by exsanguination under anesthesia. Lungs were divided for histology (fixed in 10% buffered formalin followed by conventional Hematoxylin and eosin staining) and RNA/protein analyses. Bronchoalveolar lavage (BAL) was performed by injecting 0.5 ml of PBS into the lung and gently aspirating the fluid three times. Detailed methods to qualify lung injury have been described in previous studies. As shown in FIG. 9, KLF2 mRNA-encapsulated, VCAM1-targeting lipid nanoparticles effectively delivered functional KLF2 mRNA to the inflamed mouse lung subjected to influenza H1N1 viruses, leading to the upregulation of eNOS and RAPGEF3 that are KLF2 downstream targets key to lung health. Specifically, intravenous injections of human KLF2 mRNA encapsulated, VCAM1-targeting lipid nanoparticles significantly increased human KLF2 mRNA in the mouse lung subjected to infection of H1N1 viruses. Moreover, mouse eNOS and RAPGEF3, KLF2 downstream targets key to lung health, were significantly up-regulated in the H1N1-infected lung in mice with intravenous injections of KLF2 mRNA encapsulated, VCAM1-targeting lipid nanoparticles.

Application of VCAM1-targeting lipid nanoparticles in treating lung injury in mice induced by human SARS-COV-2: The therapeutic effectiveness of the VCAM1-targeting, KLF2 mRNA-encapsulated lipid nanoparticles were tested in reducing SARS-COV-2 viral load in mice, given the key anti-viral function of KLF2 recently reported. The experiments were conducted in the BSL-3 SARS core in the Argonne National Laboratory operated by the University of Chicago. The SARS-COV-2 was propagated and the K18-hACE2 transgenic mouse line (from Jackson Lab) in which human angiotensin-converting enzyme 2 (hACE2, a functional human receptor for the coronaviruses) was knocked-in was secured. K18-hACE2 mice were intratracheally instilled either with SARS-COV-2 (105 TCID50/50 μl) or control PBS (50 μL). 24 hours after intratracheal installation, mice were intravenously injected with VCAM1-targeting KLF2 lipid nanoparticles (1 mg mRNA/kg body weight, total 100 μL). A total of 5 groups (all in 6-8 weeks old mice, both male and female) were employed in this study: 1) Intratracheal PBS (control), 2) SARS-COV-2 instilled/PBS tail vein injection, 3) SARS-COV-2 instilled/VCAM-1 targeting, control mRNA (KLF2 mutant mRNA that cannot be translated) lipid nanoparticles, 4) SARS-COV-2 virus instilled/scrambled VCAM1-targeting peptide (QAHPHVD (SEQ ID NO: 2)), KLF2 mRNA lipid nanoparticles, and 5) SARS-COV-2 instilled/VCAM-1 targeting, KLF2 mRNA lipid nanoparticles. 48 hours after tail vein injections, animals were sacrificed by exsanguination under anesthesia. Lungs were collected.

Bio-distribution of VCAM1-targeting lipid nanoparticles in vivo: To visually determine the functional distribution of this VCAM1-targeting, mRNA-encapsulated lipid nanoparticle in vivo, both the lipid nanoparticles and the overexpressed protein were tracked. mScarlet mRNAs were encapsulated in the targeted lipid nanoparticles to determine the bio-distribution of the over-expressed protein. 24 hours after injection, heart, aorta, liver, spleen, lungs, and kidneys were excised for fluorescence imaging (IVIS 200 Imaging System) and fixed/frozen for cryo-sectioning and immunofluorescence staining.

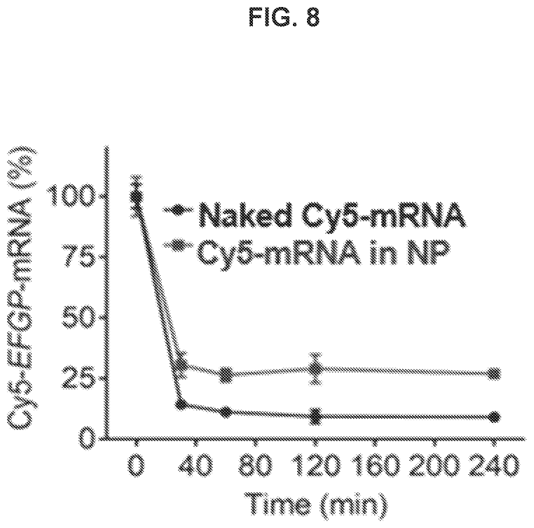

Pharmacokinetics (half-life) of KLF2 mRNA in the circulation: Preliminary results have determined the pharmacokinetics profile of Cy5-labeled mRNA (~1 kb) molecules in circulation as a function of the lipid nanoparticle in healthy B6 mice (FIG. 8). The plasma terminal half-life of Cy5-labeled KLF2 mRNA, in the naked form or encapsulated in the lipid nanoparticle, in mice was determined. Blood was collected immediately, 30 minutes, 60 minutes, 120 minutes, and 240 minutes after the injection, followed by fluorescence measurements.

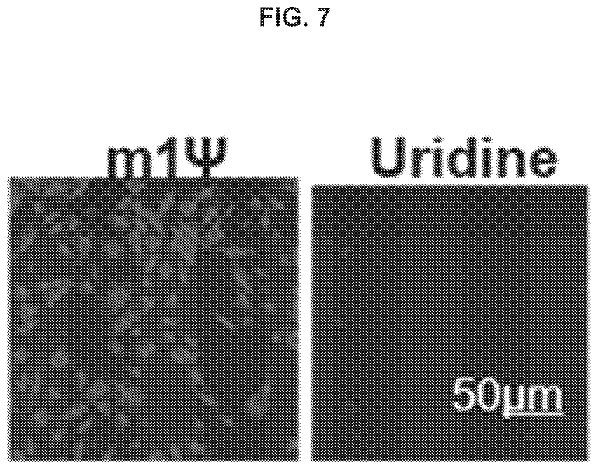

Results: The therapeutic effectiveness of the KLF2 mRNA-encapsulated, VCAM1-targeting lipid nanoparticle was demonstrated by 1) elevated KLF2 expression, 2) reduced protein and cell counts in the bronchoalveolar lavage, 3) decreased lung inflammation (by RNA/protein) and 4) lessened lung pathology (by histology). The preliminary results demonstrated that when delivered by the VCAM1-targeting lipid nanoparticle, N1-Methylpseudouridine (m1\′) incorporation significantly enhances the protein output of mScarlet mRNA in inflamed cultured PMVEC (FIG. 7). The pharmacokinetics, bio-distribution, tolerable dose, and potential toxicity/immunogenicity of the KLF2 mRNA/lipid nanoparticle was systematically determined. Preliminary data showed that VCAM1-targeting lipid nanoparticle markedly increases the half-life of Cy5-labeled mRNA in the circulation in healthy mice (FIG. 8).

### Example 3: Therapeutic Effectiveness of VCAM1-Targeting Lipid Nanoparticles Delivering KLF2 mRNA to Inflamed Endothelial Cells and Treating ARDS Induced by Influenza a Virus (A/WSN/33 [H1N1])

The therapeutic effects of VCAM1-targeting, KLF2 mRNA-encapsulated lipid nanoparticles were determined for reducing ARDS induced by SARS-COV-2 or H1N1 influenza viruses. The therapeutic effectiveness of VCAM1-targeting lipid nanoparticles in delivering KLF2 mRNA to activated endothelium was tested in mice.

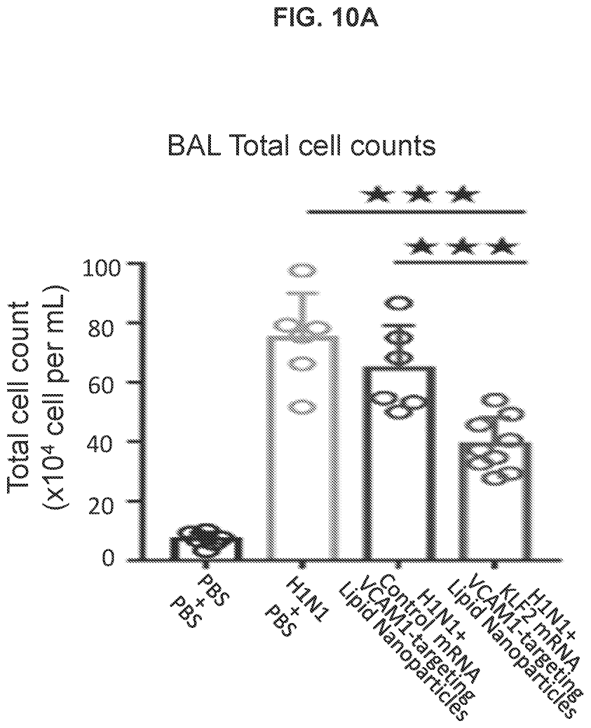

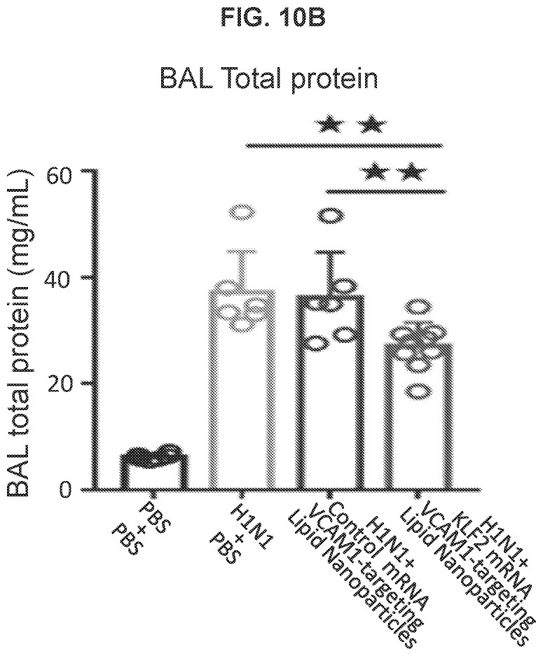

FIG. 10 demonstrates that KLF2 mRNA-encapsulated, VCAM1-targeting lipid nanoparticles significantly reduced H1N1-induced lung injury by reducing the cell count in the bronchoalveolar lavage (BAL). Intravenous injections of KLF2 mRNA encapsulated, VCAM1-targeting lipid nanoparticles significantly reduced the total cell counts in the bronchoalveolar lavage (BAL), a major surrogate of lung edema and injury. Intravenous injections of KLF2 mRNA encapsulated, VCAM1-targeting lipid nanoparticles significantly reduced H1N1-induced lung injury by reducing the total protein in the bronchoalveolar lavage (BAL).

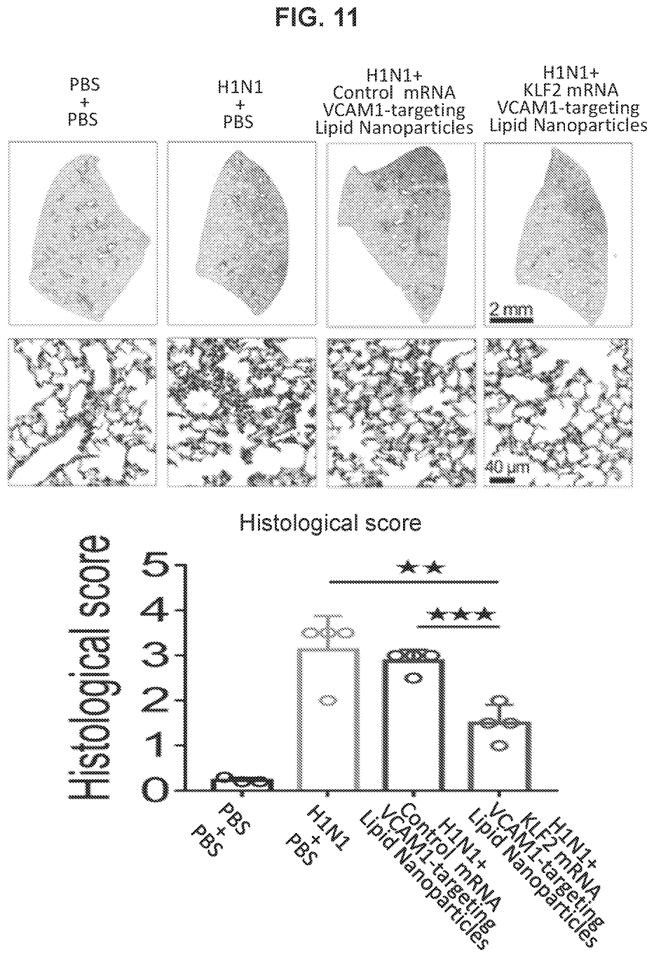

FIG. 11 shows that KLF2 mRNA-encapsulated, VCAM1-targeting lipid nanoparticles significantly reduced H1N1-induced lung pathology by reducing the lung histological (pathological) score. Mouse lung infection of influenza H1N1 viruses leads to lung injury, detected in H&E stained histological lung sections and quantified by the lung histological (pathological) score. Intravenous injections of human KLF2 mRNA encapsulated, VCAM1-targeting lipid nanoparticles significantly reduced H1N1-induced lung injury quantified by the histological (pathological) score in H&E stained lung sections. In contrast, intravenous injections of control (non-functional) mRNA encapsulated, VCAM1-targeting lipid nanoparticles had no effect on the H1N1-induced lung injury quantified by the histological (pathological) score.

### Example 4: Epithelium-Targeting Lipid Nanoparticles Delivering OAS1 mRNA to Epithelial Cells and Treating ARDS Induced by Influenza a Virus (A/WSN/33 [H1N1])

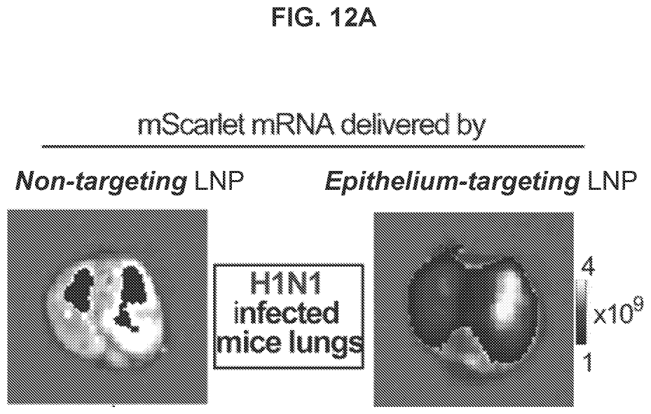

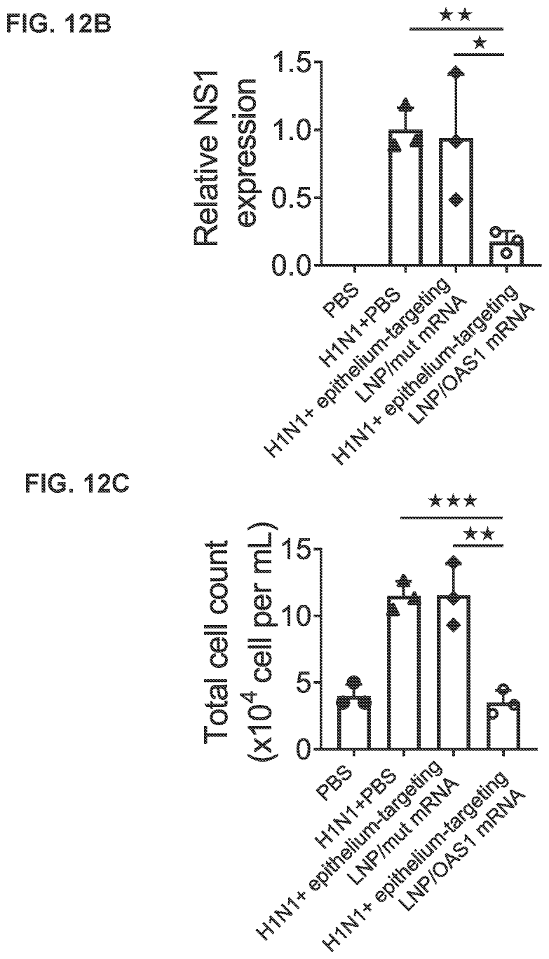

FIG. 12A demonstrates that intravenous injection of epithelium-targeting lipid nanoparticles effectively delivered functional mSCarlet mRNA to mouse ARDS lung induced by influenza A virus. The lipid nanoparticle was engineered to display the peptide CTSGTHPRC (SEQ ID NO: 8) which binds epithelium, a sequence described by Morris et al. (J. Control. 2011, 151 (1), 83-94). FIG. 12B shows that intravenous injection of OSA1 mRNA-encapsulated epithelium-targeting lipid nanoparticles significantly reduced the viral counts of influenza A virus in the mouse lung, quantified by the viral gene NS1. FIG. 12C demonstrates that intravenous injection of OSA1 mRNA-encapsulated epithelium-targeting lipid nanoparticles significantly reduced the mouse lung injury induced by influenza A virus, quantified by the total cell counts in the BAL. Data are means+SD.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

TABLE 2
Sequences.

| Sequence | SEQ ID NO: |
|---|---|
| VHPKQHR | 1 |
| QAHPHVD | 2 |
| DITWDQLWDLMK | 3 |
| CSRNLIDC | 4 |
| CTSDHAVC | 5 |
| (DSPE-PEG)-VHPKQHR | 6 |
| (DSPE-PEG)-DITWDQLWDLMK | 7 |
| CTSGTHPRC | 8 |
| DSPE-PEG- CTSGTHPRC | 9 |

What is claimed is:

1. A lipid nanoparticle, comprising:
a) a VCAM-1 targeting molecule; and
b) an RNA molecule encoding Krüppel-like Factor 2 (KLF2).

2. The lipid nanoparticle of claim 1, wherein the lipid nanoparticle comprises:
a) a polyamidoamine (PAMAM) dendrimer (G0-C14),
b) cholesterol,
c) polyethylene glycol 2000 (PEG),
d) 1,2-Distearoyl-sn-glycero-3-phosphoethanolamine-Poly(ethylene glycol) (DSPE-PEG), and
e) dioleoylphospha-tidylethanolamine (DOPE), and
wherein the lipid nanoparticle encapsulates the RNA molecule encoding KLF2.

3. The lipid nanoparticle of claim 2, wherein the DSPE-PEG comprises a PEG domain comprising PEG having an average molecular weight of about 1,000 to about 100,000 Daltons.

4. The lipid nanoparticle of either claim 2 or claim 3, wherein the VCAM-1 targeting molecule comprises a peptide comprising the amino acid sequence VHPKQHR (SEQ ID NO: 1, or DITWDQLWDLMK (SEQ ID NO: 3).

5. The lipid nanoparticle of claim 4, wherein the VCAM-1 targeted lipid nanoparticle comprises DSPE-PEG-VHPKQHR (SEQ ID NO: 6) or DSPE-PEG-DITWDQLWDLMK (SEQ ID NO: 7).

6. The lipid nanoparticle of claim 1, wherein the RNA molecule encoding KLF2 is mRNA.

7. The lipid nanoparticle of claim 6, wherein the KLF2 mRNA is a m1-substituted KLF2 mRNA.

8. The lipid nanoparticle of any one of claims 1-7, wherein the lipid nanoparticle encapsulates about 0.01 μM to about 100 μM of the RNA molecule encoding KLF2.

9. The lipid nanoparticle of any one of claims 1-8, wherein the molar ratio of:
a) the polyamidoamine (PAMAM) dendrimer (G0-C₁₄) to DSPE-PEG is about 2:1 to about 15:1;
b) cholesterol to DSPE-PEG is about 15:1 to about 40:1; and
c) DOPE to DSPE-PEG is about 15:1 to about 30:1.

10. A pharmaceutical composition, comprising:
a) a therapeutically effective amount of a lipid nanoparticle comprising a VCAM-1 targeting molecule and an RNA molecule encoding KLF2; and
b) a pharmaceutically acceptable carrier, solvent, adjuvant, and/or diluent.

11. The pharmaceutical composition of claim 10, wherein the pharmaceutical composition is formulated for inhalation, insufflation, oral, intravenous, topical, ocular, buccal, systemic, nasal, injection, transdermal, rectal, or vaginal administration.

12. The pharmaceutical composition of either claim 10 or claim 11, wherein the pharmaceutical composition is formulated for inhalation or insufflation.

13. A pharmaceutical composition for pulmonary delivery of RNA comprising:
a) a lipid nanoparticle comprising
i) a VCAM-1 targeting molecule; and
ii) an RNA molecule encoding KLF2; and
b) a pharmaceutically acceptable carrier,
wherein the composition is formulated such that once administered to the lung, it results in the delivery of the RNA molecule to a lung cell.

14. A method of treating a lung disorder in a subject, comprising:
a) administering to the subject a therapeutically effective amount of a pharmaceutical composition comprising a lipid nanoparticle comprising a VCAM-1 targeting molecule and an RNA molecule encoding KLF2, wherein the lipid nanoparticle is preferentially targeted to inflamed endothelial cells associated with the lung disorder; and
b) reducing inflammation at the site of the inflamed endothelial cells.

15. The method of claim 14, wherein the lung disorder comprises one or more of Acute Respiratory Distress Syndrome (ARDS), Acute Lung Injury (ALI), or Ventilator-Induced Lung Injury (VILI).

16. The method of either claim 14 or claim 15, wherein the method results in one or more of an increase of at least about 2 fold to at least about 1000 fold of the RNA encoding KLF2 compared to a control, an increase of at least about 2 fold to at least about 200 fold of KLF2 protein compared to a control, or a reduction in viral titers compared to a control at the site of the inflamed endothelial cells.

17. The method of any one of claims 14-16, wherein the probability of survival of the subject is at least about 10% greater than an expected probability of survival without administration of the pharmaceutical composition.

18. A lipid nanoparticle, comprising:
a) a epithelium targeting molecule; and
b) an RNA molecule encoding 2′-5′-oligoadenylatesynthetase 1 (OAS1)

19. The lipid nanoparticle of claim 1, wherein the lipid nanoparticle comprises:
a) a polyamidoamine (PAMAM) dendrimer (G0-C₁₄),
b) cholesterol,
c) polyethylene glycol 2000 (PEG),
d) 1,2-Distearoyl-sn-glycero-3-phosphoethanolamine-Poly(ethylene glycol) (DSPE-PEG), and
e) dioleoylphospha-tidylethanolamine (DOPE), and
wherein the lipid nanoparticle encapsulates the RNA molecule encoding OAS1.

20. The lipid nanoparticle of claim 19, wherein the DSPE-PEG comprises a PEG domain comprising PEG having an average molecular weight of about 1,000 to about 100,000 Daltons.

21. The lipid nanoparticle of either claim 19 or claim 20, wherein the epithelium targeting molecule comprises a peptide comprising the amino acid sequence CTSGTHPRC (SEQ ID NO: 8).

22. The lipid nanoparticle of claim 21, wherein the epithelium targeted lipid nanoparticle comprises DSPE-PEG-CTSGTHPRC (SEQ ID NO: 9).

23. The lipid nanoparticle of claim 18, wherein the RNA molecule encoding OAS1 is mRNA.

24. The lipid nanoparticle of claim 23, wherein the OAS1 mRNA is a m1Ψ-substituted OAS1 mRNA.

25. The lipid nanoparticle of any one of claims 18-24, wherein the lipid nanoparticle encapsulates about 0.01 μM to about 100 μM of the RNA molecule encoding OAS1.

26. The lipid nanoparticle of any one of claims 18-25, wherein the molar ratio of:
a) the polyamidoamine (PAMAM) dendrimer (G0-C₁₄) to DSPE-PEG is about 2:1 to about 15:1;
b) cholesterol to DSPE-PEG is about 15:1 to about 40:1; and
c) DOPE to DSPE-PEG is about 15:1 to about 30:1.

27. A pharmaceutical composition, comprising:
a) a therapeutically effective amount of a lipid nanoparticle comprising a epithelium targeting molecule and an RNA molecule encoding OAS1; and
b) a pharmaceutically acceptable carrier, solvent, adjuvant, and/or diluent.

28. The pharmaceutical composition of claim 27, wherein the pharmaceutical composition is formulated for inhalation, insufflation, oral, intravenous, topical, ocular, buccal, systemic, nasal, injection, transdermal, rectal, or vaginal administration.

29. The pharmaceutical composition of either claim 27 or claim 28, wherein the pharmaceutical composition is formulated for inhalation or insufflation.

30. A pharmaceutical composition for pulmonary delivery of RNA, comprising:
a) a lipid nanoparticle comprising
i) a epithelium targeting molecule; and
ii) an RNA molecule encoding OAS1; and
b) a pharmaceutically acceptable carrier,
wherein the composition is formulated such that once administered to the lung, it results in the delivery of the RNA molecule to a lung cell.

31. A method of treating a lung disorder in a subject, comprising:
a) administering to the subject a therapeutically effective amount of a pharmaceutical composition comprising a lipid nanoparticle comprising a epithelium targeting molecule and an RNA molecule encoding OAS1, wherein the lipid nanoparticle is preferentially targeted to inflamed endothelial cells associated with the lung disorder; and
b) reducing inflammation at the site of the inflamed endothelial cells.

32. The method of claim 31, wherein the lung disorder comprises one or more of Acute Respiratory Distress Syndrome (ARDS), Acute Lung Injury (ALI), or Ventilator-Induced Lung Injury (VILI).

33. The method of either claim 31 or claim 32, wherein the method results in one or more of an increase of at least about 2 fold to at least about 1000 fold of the RNA encoding OAS1 compared to a control, an increase of at least about 2 fold to at least about 2000 fold of OAS1 protein compared to a control, or a reduction in viral titers compared to a control at the site of the inflamed endothelial cells.

34. The method of any one of claims 31-33, wherein the probability of survival of the subject is at least about 10% greater than an expected probability of survival without administration of the pharmaceutical composition.
